(19) United States
(12) Patent Application Publication
(10) Pub. No.: US 2013/0202625 A1
(43) Pub. Date: Aug. 8, 2013

(54) USE OF HUMAN ERYTHROCYTES FOR PREVENTION AND TREATMENT OF CANCER DISSEMINATION AND GROWTH

(71) Applicants: Dmitri Simberg, San Diego, CA (US); Guixin Shi, San Diego, CA (US)

(72) Inventors: Dmitri Simberg, San Diego, CA (US); Guixin Shi, San Diego, CA (US)

(73) Assignee: The Regents Of The University of Californa, Oakalnd (CA)

(21) Appl. No.: 13/522,909

(22) PCT Filed: Jan. 20, 2011
(86) PCT No.: PCT/US11/21894

§ 371 Date: Oct. 12, 2012

Related U.S. Application Data
(60) Provisional application No. 61/297,124, filed on Jan. 21, 2010.

(51) Int. Cl. A61K 35/18

(52) U.S. Cl. CPC A61K 35/18
USPC 424/178.1; 424/93.73; 435/325

(57) ABSTRACT

The technology relates in part to methods of preventing and treating diseases and conditions associated with cancer, including methods, compositions, and kits used for preventing and treating cancer dissemination and growth.

### RELATED APPLICATION(S)

This patent application is a national stage of International Patent Application No. PCT/US2011/021894, filed Jan. 20, 2011, entitled USE OF HUMAN ERYTHROCYTES FOR PREVENTION AND TREATMENT OF CANCER DISSEMINATION AND GROWTH, naming Dmitri Simberg and Guixin Shi as inventors, and designated by Attorney Docket No. UCS-1002-PC, which claims priority to U.S. Provisional Application No. 61/297,124, filed Jan. 21, 2010, entitled Use of Human Erythrocytes for Prevention and Treatment of Metastatic Cancer Dissemination and Growth, naming Dmitri Simberg as inventor, and designated by Attorney Docket No. UCS-1002-PV. The entire contents of which are incorporated herein by reference in their entirety.

### FIELD

The technology relates in part to methods of preventing and treating diseases and conditions associated with metastatic cancer, including methods, compositions, and kits used for preventing and treating cancer dissemination and growth.

### BACKGROUND

Cancer metastasis is caused by populations of aggressive tumor cells that detach from the primary tumor, enter the blood and the lymph system, and finally colonize distant organs. The formation of new blood vessels (angiogenesis) is crucial for the growth and persistence of primary solid tumors and their metastases, and it has been assumed that angiogenesis is also required for metastatic dissemination, because an increase in vascular density will allow easier access of tumor cells to the circulation. In fact, angiogenesis indicates poor prognosis and increased risk of metastasis in many cancer types, including breast cancer [11].

Metastatic breast cancer is an incurable disease with a median survival of approximately 2 to 3 years. Death, and most of the complications associated with breast cancer, are due to metastasis developing in regional lymph nodes and in distant organs, including bone, lung, liver, and brain [1]. Aggressive systemic chemotherapy is necessary in case of invasive breast cancer due to distant metastatic spread early at the time of diagnosis [2]. For most patients, these treatments are only partially effective and result in only limited prolongation of survival [3, 4][5].

There are two major routes for breast tumor dissemination: lymphatic vessels and blood vessels [1]. Hematogenous spread occurs at a later time and results in more distant metastases. A tumor cell that leaves the primary tumor and inravasates must survive within the circulation, become arrested in capillaries or venules of other organs, extravasate and adapt to the newly colonized milieu to form the new tumor [6, 7].

The levels of circulating tumor cells in peripheral blood were shown to inversely correlate with survival in advanced breast cancer patients [8-10]. Some of the metastatic cells populate the bone marrow and constitute a pool of the metastatic cells regardless of the main tumor [8], and bone marrow transplantation has been practiced in order to remove metastatic cells.

Red blood cells, which circulate in peripheral blood, have been discussed as a vehicle for drug delivery and for their use in imaging [28]. Drugs have been entrapped in erythrocytes for delivery as cellular carriers. [28, 29] Avidin-biotin bridges have also been used for reversible membrane binding of proteins and other biopharmaceuticals, and antigens. [28]

There is a need for preventing and slowing the growth of cancer, preventing the circulation of tumor cells, and reducing the levels of circulating tumor cells in cancer patients. There is also a need for inhibiting or slowing angiogenesis in order to block or reduce the growth of primary solid tumors, and to reduce metastatic dissemination.

### SUMMARY

The technology relates in part to methods of treating and preventing diseases and conditions associated with cancer metastasis and with a primary tumor, such as, for example, breast cancer metastasis, by blocking the circulation of metastatic cancer cells, and by blocking angiogenesis, such as, for example, capturing circulating endothelial progenitors that are recruited to the tumor, or by physically blocking (infarction) of the capillaries of the tumor or the metastasis.

Although many new therapeutic approaches for cancer metastasis focus on the inhibition of molecular pathways of the metastatic invasion and growth, the present application relates to physically blocking metastasis and angiogenesis. The metastatic process is physically interrupted by incorporating tumor- and angiogenesis-specific ligands such as antibodies, single chain antibodies, small molecules, and peptides into the plasma membrane of erythrocytes.

Red blood cells have potential for use as therapeutics as they are easily retrieved from a patient, non-immunogenic, and are biologically designed to navigate the microcirculation, including tortuous tumor vasculature. [30, 31] For example, autologous erythrocytes may be linked to tumor vasculature-targeted antibodies [32], and a targeted therapy and diagnostic platform can be developed whereby the modified cells are re-injected into a patient and accumulate in the tumor circulation. The modified cells may be designed to deliver a chemotherapeutic drug payload to tumor capillaries. The cells may also be used for diagnostics and imaging by incorporating fluorophores or ultrasound contrast agents within the modified erythrocytes.

These engineered red blood cells, or erythrocytes, may be administered at different stages of the metastatic dissemination process. For example, the cells could be used before, during and after surgery to prevent dissemination of the tumor cells; as an adjunct to chemotherapy and radiotherapy; or during advanced metastatic disease when no other options are available.

Thus, provided herein are methods for inhibiting the dissemination of cancer cells in a patient, comprising contacting the cells with a red blood cell linked to a cancer cell-specific ligand. Also provided herein are methods for preventing or treating metastatic cancer dissemination in a patient comprising administering to the patient a red blood cell linked to a metastatic cancer cell-specific ligand.

In some embodiments, the cancer cells are primary cancer cells. In some embodiments, the cancer cells are metastatic cancer cells. In some embodiments, the cell-specific ligand is an antibody. In some embodiments, the cell-specific ligand is a peptide or a small molecule. In some embodiments, the ligand is conjugated to a lipid, a lipopeptide, or a transmembrane protein domain, and the conjugated ligand is incorporated into the cell membrane of the red blood cell. In some embodiments, the ligand is an antibody that binds to an antigen selected from the group consisting of prostate specific membrane antigen, carcinoembryonic antigen, integrin alpha v beta 3, integrin alpha v beta 5, EpCAM, CD133, nucleolin, VEGF receptor 1 and VEGF receptor 2. In some embodiments, the ligand is covalently linked to a molecule on the cell membrane of the red blood cell. In some embodiments, the ligand is linked to the red blood cell membrane using photoactivatable chemistry. In some embodiments, the ligand is conjugated to a lipid. In some embodiments, the lipid is a non-phospholipid. In some embodiments, the lipid is selected from the group consisting of acyl, alkyl, ceramides, gangliosides, sphingosines, sterols, and sphyngomyelin. In some embodiments, the lipid is Dim-23, DSPE, or DEPE. In some embodiments, the lipid is conjugated to a label. In some embodiments, the lipid has 12 to 22 carbons. In some embodiments, the lipid is single chained. In some embodiments, the lipid is multiple-chained. In some embodiments, one or more of the lipid chains is monounsaturated, in some embodiments, one or more of the lipid chains is polyunsaturated. In some embodiments, the lipid chain is mono or polyunsaturated. In some embodiments, the lipid is an 18 carbon lipid. In some embodiments, the ligand is conjugated to the lipid, lipopeptide, or transmembrane protein domain by a PEG linker. In some embodiments, the red blood cell is linked to an immunomodulating signal. In some embodiments, the immunomodulating signal is a FAS ligand, or a FAC receptor antibody. In some embodiments, the patient is human. In some embodiments, the cancer cells and the red blood cells linked to ligands form cell complexes.

Also provided herein are methods for inhibiting the growth of neovasculature in a patient comprising administering to the patient a red blood cell linked to an angiogenic cell targeting ligand. Also provided are methods for inhibiting the growth of neovasculature in a patient, comprising administering to the patient a red blood cell linked to an endothelial progenitor cell targeting ligand. Also provided are methods for inhibiting the growth of neovasculature in a patient comprising administering to the patient a red blood cell linked to an angiogenic cell targeting ligand.

In some embodiments, the ligand is an antibody. In some embodiments, the ligand is a peptide or a small molecule. In some embodiments, the ligand adheres to early angiogenic capillaries. In some embodiments, the neovasculature is associated with a tumor. In some embodiments, the neovasculature is associated with a metastatic tumor. In some embodiments, the growth of the tumor is inhibited after administering the red blood cell to the patient.

Also provided are compositions comprising a red blood cell linked to a cancer cell-specific ligand. In some embodiments the ligand is an antibody. In some embodiments, the ligand is a small molecule or peptide.

Also provided are compositions comprising a red blood cell linked to an anti-angiogenic cell antibody. In some embodiments, the antibody adheres to early angiogenic capillaries.

Also provided are compositions comprising a red blood cell linked to an endothelial progenitor cell targeting ligand.

In some embodiments, the red blood cell is type A, B, AB, or O.

Also provided are kits comprising a red blood cell linked to a cancer cell-specific ligand. Also provided are kits comprising a red blood cell linked to an anti-angiogenic cell antibody. Also provided are kits comprising a red blood cell linked to an endothelial progenitor cell targeting ligand. In some embodiments, the kits further comprise instructions. In some embodiments, the red blood cell is type A, B, AB, or O.

Also provided are kits comprising a metastatic cell-specific ligand and a composition for linking the ligand to a red blood cell. Also provided are kits comprising an angiogenic cell targeting ligand and a composition for linking the ligand to a red blood cell. Also provided are kits comprising an endothelial progenitor cell targeting ligand and a composition for linking the ligand to a red blood cell.

In some embodiments, the ligand is an antibody. In some embodiments, the ligand is a small molecule or a peptide. In some embodiments, the kits further comprise instructions for linking the ligand to the red blood cell. In some embodiments, the composition for linking the ligand to the red blood cell is selected from the group consisting of lipid, lipopeptide, and transmembrane protein domain. In some embodiments, the composition for linking the ligand to the red blood cell further comprises a PEG linker.

Also provided are methods for inhibiting the dissemination of a blood borne pathogen in the blood stream comprising contacting the pathogen with a red blood cell linked to a pathogen-specific ligand. In some embodiments the ligand is an antibody. In some embodiments, the ligand is a small molecule or peptide. In some embodiments, the pathogen is a bacteria. In some embodiments, the pathogen is a virus.

Also provided are methods for linking a metastatic cell-specific ligand, an angiogenic cell targeting ligand, or an endothelial progenitor cell targeting ligand to a red blood cell, comprising providing a metastatic cell specific ligand, and a PEG linker, wherein the PEG linker is linked to a molecule selected from the group consisting of a lipid, a lipoprotein, and a transmembrane domain; linking the PEG linker to the ligand to obtain a linked ligand; and conjugating the linked ligand to a red blood cell. In some embodiments, the method further comprises linking the PEG linker to the molecule selected from the group consisting of a lipid, a lipoprotein, and a transmembrane domain. In some embodiments, the linked ligand is conjugated to the red blood cell by incubating the linked ligand with the red blood cell in solution. In some embodiments, the ligand is linked to the PEG linker by modifying the linker with sulfhydryl groups and coupling the sulfhydryl group modified ligand to the PEG linker.

Certain embodiments are described further in the following description, examples, and claims.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1 is a graphical depiction of the proposed interaction of modified red blood cells with metastatic or angiogenic cells.

FIG. 2 depicts binding of modified red blood cells to integrin-expressing B16/F1 tumor cells grown in microsopy chambers. The black dots on the left image are red blood cells that adhered to the cells.

FIG. 3 depicts IgG coated red blood cells in a blood sample obtained from mice.

FIG. 4 presents a diagram of a red blood cell modification strategy.

FIG. 5 presents the chemical structures, formulas, and molecular weights of sample lipids.

FIG. 6 presents an example of a chart that may be used to record the results of a red blood cell stability test in mice.

FIG. 7 presents graphs of red blood cell in vivo stability tests.

FIG. 8 presents a chart of the lipid-PEG-IgG pharmacokinetic parameters in vivo, measured by FACS.

FIG. 9 presents photos of blood smears obtained from mice after injection of modified red blood cells.

FIG. 10 presents the results of a DSPE-PEG-IgG in vivo stability test, measured by FACS.

FIG. 11 presents the results of a DSPE-PEG-IgG in vivo stability test, measured by FACS.

FIG. 12 presents photos of DSPE-PEG-IgG red blood cells in mouse blood at 37 degrees Celsius.

FIG. 13 presents photos of an in vivo stability test.

FIG. 14 presents photos of an EpCAM/A549 binding test.

FIGS. 15 and 16 present photos from an EpCAM/A549 binding test.

### DETAILED DESCRIPTION

Chemically modified, or engineered erythrocytes may be used to prevent and treat dissemination and colonization of primary cancer cells and metastatic tumor cells in the body. Erythrocytes may be taken from blood and “reprogrammed” to be able to specifically adhere to cells, such as, for example, blood borne metastatic cells, to the inner lining of metastatic blood vessels (endothelium), to primary cancer cells, or to vascular and endothelial stem cells that are recruited from bone marrow. Once injected back into the body, the red blood cells will continuously travel in the bloodstream until they encounter metastatic cells or metastatic blood vessels. This will reduce the capacity of the tumor cells to colonize the organs and also will stop the blood supply in the already existing metastasis. This method focuses on the physical interruption of the metastatic process by formation of cell complexes of coated erythrocytes with circulating metastatic cells, angiogenic endothelium and/or endothelial progenitor cells. A cell complex may comprise at least 2, 3, 4, 5, 6, 7, 8, 9, 10, 11, 12, 13 14, 15, 20, 25, 30, 35, 40, 45, or 50 coated red blood cells adhering to a tumor cell.

The method takes advantages of the long circulating lifetime (120 days) of the erythrocyte, with a half-life of between about 3 hours and about 30 days, and their abundance (2-3×10¹³ in adult humans). Autologous or compatible erythrocytes are coated with antibodies against markers of circulating metastatic cells, angiogenic endothelium and/or endothelial progenitor cells.

In one embodiment, erythrocytes coated with tumor cell-specific antibodies may be used to prevent and treat metastasis by, for example, capturing and neutralizing the circulating metastatic cells. In another embodiment, the coated erythrocytes may be used to sequester tumor cells in the reticuloendothelial system to prevent their entry to the organs and their colonization.

Without limiting the embodiments to a particular method of action, the modified red blood cell-metastatic cell complexes may circulate in the bloodstream and eventually be trapped and, for example, destroyed in the reticulo-endothelial system, such as, for example, in the liver or spleen.

In another embodiment, erythrocytes coated with angiogenesis-specific antibodies may be used to block the growth of neovasculature and reduce the blood supply to tumors by, for example, physically adhering to the early angiogenic capillaries, and plugging them, thereby stopping blood flow. In another embodiment, erythrocytes coated with endothelial progenitor cell-specific antibodies may be used to, for example, inhibit the growth of neovasculature and reduce the blood supply to tumors. Endothelial progenitor cells have been implicated in neovascularization of tumors. (33) In addition, modified red blood cells may be designed to target circulating stem cells derived from bone marrow, and to target endothelial progenitor cells, by modifying the red blood cell with, for example, CD133 ligand. (33, 34) In other embodiments, erythrocytes may be coated with anti-angiogenic ligands such as antibodies that block tumor blood vessels and tumor associated vasculature. The methods may be used to inhibit the blood supply to primary tumors as well as secondary tumors, such as metastatic tumors that develop due to metastasis of the primary tumor. By blocking the growth of the neovasculature, the blood supply to the tumors may be reduced or blocked so that the tumor eventually shrinks or is destroyed.

The proposed method could benefit many categories of cancer patients, such as, for example, cancer patients having melanoma, adenocarcinoma, squamous cell carcinoma, adenosquamous cell carcinoma, thymoma, lymphoma, sarcoma, lung cancer, liver cancer, non-Hodgkin's lymphoma, Hodgkin's lymphoma, leukemias, uterine cancer, breast cancer, prostate cancer, ovarian cancer, pancreatic cancer, colon cancer, multiple myeloma, neuroblastoma, NPC, bladder cancer, cervical cancer, and glioblastoma by improving the quality of treatment and prognosis. The treatment could be employed at different stages of the metastatic dissemination process. For example, the method could be used before, during and after surgery; as an adjunct to chemotherapy and radiotherapy; or during advanced metastatic disease when no other options are available.

In some embodiments, the red blood cells may be linked to, or coated with, both the angiogenic cell targeting ligand and the cancer cell-specific ligand. In yet other embodiments, both red blood cells coated with or linked to angiogenic cell targeting ligands, and red blood cells linked to or coated with cancer cell-specific ligands, may be administered to the patient.

In yet another embodiment, the red blood cells may be linked to, or coated with ligands specific for blood borne pathogens, for the treatment of blood borne diseases. Blood borne diseases, such as, for example pathogens, for example, bacteria and viruses may be contacted with red blood cells that are coated with ligands specific for the bacteria or viruses, thereby neutralizing the pathogen. The red blood cell may further comprise an immunomodulating agent.

In another embodiment, the various red blood cell coating components may be assembled into kits with, for examples, instructions for the preparation of coated red blood cells at a treatment site, using autologous or compatible red blood cells. Additionally, coated RBCs of type A, B, AB, or O may be prepared in kits and supplied as ready-to-use therapeutics. The kits of the present technology may also comprise one or more of the components in any number of separate containers, packets, tubes, vials, microtiter plates and the like, or the components may be combined in various combinations in such containers.

The components of the kit may, for example, be present in one or more containers, for example, all of the components may be in one container. The components may, for example, be lyophilized, freeze dried, or in a stable buffer.

The kits of the present technology may also comprise instructions for performing one or more methods described herein and/or a description of one or more compositions or reagents described herein. Instructions and/or descriptions may be in printed form and may be included in a kit insert. A kit also may include a written description of an Internet location that provides such instructions or descriptions.

By cancer cell-specific or cancer cell blocking ligand is meant a protein, small molecule, polypeptide, or peptide, including for examples, antibodies or single chain antibodies, that binds to a cancer cell, for example, one that specifically binds to a specific primary cancer cell or metastatic cell marker. By angiogenic cell targeting or neovasculature targeting ligand or antibody is meant a ligand or antibody that binds to angiogenic cells, for example, one that specifically binds to a specific angiogenic cell marker. The binding vehicle of the coated red blood cells and targeted cells, for example metastatic cells or angiogenic cells, can be any ligand/receptor combination and is not limited to antigen/antibody. By endothelial progenitor cell targeting ligand is meant a ligand or antibody that binds to endothelial progenitor cells, for example, one that specifically binds to a specific endothelial progenitor cell marker.

In yet another embodiment, red blood cells may be coated with a ligand that prevents or reduces the dissemination of blood borne infections in the blood.

By inhibiting is meant reducing the number of circulating primary cancer or metastatic cells, the growth rate of the primary cancer cell metastatic cell population, the number and/or size of metastases, the number of angiogenic cells, the growth rate of the angiogenic cell population, or reducing the growth of neovasculature. By inhibiting, or reducing the growth, for example, is meant a reduction in number, volume, size, or other metric by about 5, 10, 15, 20, 25, 30, 35, 40, 45, 50, 60, 70, 80, or 90%.

By coated or linked is meant that the red blood cell is engineered to be coated with, or to incorporate into its cell membrane, a ligand, such as, for example, an antibody; the ligand may also be chemically attached to the cell membrane. The technology includes methods of coating or linking the ligand to the red blood cell, for example, but not limited to, methods of linking via a lipid anchor, transmembrane protein domain anchor, lipopeptide anchor, or through covalent chemistry. By ligand is meant any substance that forms a complex with a biomolecule by, for example, binding to a site on the target biomolecule. Examples of ligands include, but are not limited to, proteins, polypeptides, peptides, lipoproteins, Lipopeptides, or any other molecule that may bind to a biomolecule. Examples include, but are not limited to, antibodies that bind to prostate specific membrane antigen, carcinoembryonic antigen, integrin alpha v beta 3, EGF receptor family, integrin alpha v beta 5, EpCAM, CD133, nucleolin, VEGF receptor 1, VEGF receptor 2, and cyclic RGD peptide, phage displayed peptides, dendrimers.

The use of the term erythrocyte, red blood cell, and RBC is interchangeable for purposes of this application.

The term “cancer” as used herein is defined as a hyperproliferation of cells whose unique trait—loss of normal controls—results in unregulated growth, lack of differentiation, local tissue invasion, and metastasis. Examples include but are not limited to, melanoma, non-small cell lung, small-cell lung, lung, hepatocarcinoma, leukemia, retinoblastoma, astrocytoma, glioblastoma, gum, tongue, neuroblastoma, head, neck, breast, pancreatic, prostate, renal, bone, testicular, ovarian, mesothelioma, cervical, gastrointestinal, lymphoma, brain, colon, sarcoma or bladder. Forms of cancer that result in circulating metastatic cells are contemplated herein.

The term “hyperproliferative disease” is defined as a disease that results from a hyperproliferation of cells. Exemplary hyperproliferative diseases include, but are not limited to cancer or autoimmune diseases. Other hyperproliferative diseases may include vascular occlusion, restenosis, atherosclerosis, or inflammatory bowel disease. These are also contemplated to be targeted by the modified red blood cells discussed herein.

As used herein, the term “polypeptide” is defined as a chain of amino acid residues, usually having a defined sequence. As used herein the term polypeptide is interchangeable with the terms “peptides” and “proteins”.

The term “subject” or patient as used herein includes, but is not limited to, an organism or animal; a mammal, including, e.g., a human, non-human primate (e.g., monkey), mouse, pig, cow, goat, rabbit, rat, guinea pig, hamster, horse, monkey, sheep, or other non-human mammal; a non-mammal, including, e.g., a non-mammalian vertebrate, such as a bird (e.g., a chicken or duck) or a fish, and a non-mammalian invertebrate.

As used herein, the terms “treatment”, “treat”, “treated”, or “treating” refer to prophylaxis and/or therapy. When used with respect to an infectious disease, for example, the term refers to a prophylactic treatment which increases the resistance of a subject to infection with a pathogen or, in other words, decreases the likelihood that the subject will become infected with the pathogen or will show signs of illness attributable to the infection, as well as a treatment after the subject has become infected in order to fight the infection, e.g., reduce or eliminate the infection or prevent it from becoming worse.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having”, “including”, “containing” and “comprising” are interchangeable and one of skill in the art is cognizant that these terms are open ended terms. Still further, the use of the word “or” as in “a or b” is meant to include either a or b, or both a and b.

### Formulations and Routes for Administration to Patients

Where clinical applications are contemplated, it will be necessary to prepare pharmaceutical compositions. One may generally desire to employ appropriate salts and buffers to render delivery of the modified red blood cells. The phrase “pharmaceutically or pharmacologically acceptable” refers to molecular entities and compositions that do not produce adverse, allergic, or other untoward reactions when administered to an animal or a human. A pharmaceutically acceptable carrier includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the cells, its use in therapeutic compositions is contemplated. Supplementary active ingredients also can be incorporated into the compositions.

Upon formulation, the modified red blood cell compositions will be administered in a manner compatible with the dosage formulation and in such amount as is therapeutically effective. Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by FDA Office of Biologics standards.

An effective amount of the pharmaceutical composition would be the amount that achieves this selected result of inhibiting metastatic cell circulation, or inhibiting angiogenesis or neovasculature formation.

The effective amount for any particular application can vary depending on such factors as the disease or condition being treated, the particular composition being administered, the size of the subject, and/or the severity of the disease or condition. One of ordinary skill in the art can empirically determine the effective amount of a particular composition presented herein without necessitating undue experimentation.

In certain embodiments, anti-cancer agents may be used in combination with the present methods. An “anti-cancer” agent is capable of negatively affecting cancer in a subject, for example, by killing one or more cancer cells, inducing apoptosis in one or more cancer cells, reducing the growth rate of one or more cancer cells, reducing the incidence or number of metastases, reducing a tumor's size, inhibiting a tumor's growth, reducing the blood supply to a tumor or one or more cancer cells, promoting an immune response against one or more cancer cells or a tumor, preventing or inhibiting the progression of a cancer, or increasing the lifespan of a subject with a cancer. Anti-cancer agents include, for example, chemotherapy agents (chemotherapy), radiotherapy agents (radiotherapy), a surgical procedure (surgery), immune therapy agents (immunotherapy), genetic therapy agents (gene therapy), hormonal therapy, other biological agents (biotherapy) and/or alternative therapies.

In further embodiments antibiotics can be used in combination with the pharmaceutical composition to treat and/or prevent an infectious disease. Such antibiotics include, but are not limited to, amikacin, aminoglycosides (e.g., gentamycin), amoxicillin, amphotericin B, ampicillin, antimonials, atovaquone sodium stibogluconate, azithromycin, capreomycin, cefotaxime, cefoxitin, ceftriaxone, chloramphenicol, clarithromycin, clindamycin, clofazimine, cycloserine, dapsone, doxycycline, ethambutol, ethionamide, fluconazole, fluoroquinolones, isoniazid, itraconazole, kanamycin, ketoconazole, minocycline, ofloxacin), para-aminosalicylic acid, pentamidine, polymixin definsins, prothionamide, pyrazinamide, pyrimethamine sulfadiazine, quinolones (e.g., ciprofloxacin), rifabutin, rifampin, sparfloxacin, streptomycin, sulfonamides, tetracyclines, thiacetazone, trimethaprim-sulfamethoxazole, viomycin or combinations thereof.

More generally, such an agent would be provided in a combined amount with the expression vector effective to kill or inhibit proliferation of a cancer cell and/or microorganism. This process may involve contacting the cell(s) with an agent(s) and the pharmaceutical composition at the same time or within a period of time wherein separate administration of the pharmaceutical composition and an agent to a cell, tissue or organism produces a desired therapeutic benefit. This may be achieved by contacting the cell, tissue or organism with a single composition or pharmacological formulation that includes both the pharmaceutical composition and one or more agents, or by contacting the cell with two or more distinct compositions or formulations, wherein one composition includes the pharmaceutical composition and the other includes one or more agents.

The terms “contacted” and “exposed,” when applied to a cell, tissue or organism, are used herein to describe the process by which an RBC or ligand is delivered to a target cell, tissue or organism or are placed in direct juxtaposition with the target cell, tissue or organism.

The administration of the pharmaceutical composition may precede, be co-current with and/or follow the other agent(s) by intervals ranging from minutes to weeks. In embodiments where the pharmaceutical composition and other agent(s) are applied separately to a cell, tissue or organism, one would generally ensure that a significant period of time did not expire between the times of each delivery, such that the pharmaceutical composition and agent(s) would still be able to exert an advantageously combined effect on the cell, tissue or organism. For example, in such instances, it is contemplated that one may contact the cell, tissue or organism with two, three, four or more modalities substantially simultaneously (i.e., within less than about a minute) with the pharmaceutical composition. In other aspects, one or more agents may be administered within of from substantially simultaneously, about 1 minute, to about 24 hours to about 7 days to about 1 to about 8 weeks or more, and any range derivable therein, prior to and/or after administering the modified cells. Yet further, various combination regimens of the pharmaceutical composition presented herein and one or more agents may be employed.

### EXAMPLES

The examples set forth below illustrate certain embodiments and do not limit the technology.

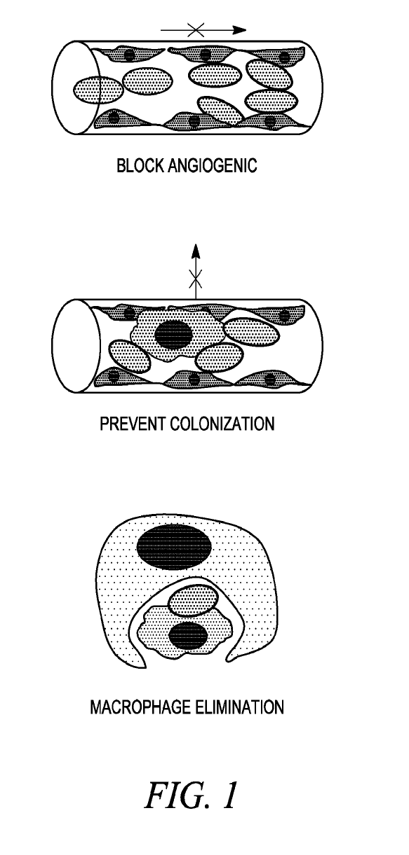

In certain examples, multiple aspects of metastatic spread may be targeted using long-circulating multifunctional red blood cells (RBCs) coated with antibodies against markers of circulating metastatic cells, angiogenic endothelium and endothelial progenitor cells: EpCAM and alpha v beta 3 integrin [12, 13]. The antibody-modified RBCs may systemically prevent or decrease the metastatic process by performing one or many of the following functions (FIG. 1): (a) capture and neutralize tumor cells in the circulation and in bone marrow; (b) sequester the tumor cells in reticuloendothelial system; (c) capture and neutralize endothelial progenitor cells; (d) block the growth of neovasculature and blood supply by physically adhering to the early angiogenic endothelium. In certain embodiments, the red blood cell may carry immunomodulating signals that enhance an immune response against the bound tumor cells. Examples of immunomodulating signals include, but are not limited to, antibodies against the FAS receptor, or the FAS ligand.

### Example 1

### Materials and Methods Used in the Foregoing Examples

Materials and methods that may be used in the methods of the technology are presented herein.

### Materials

DSPE (1,2-distearoyl-sn-glycero-3-phosphoethanolamine) was obtained from Avanti Polar Lipids Inc. DEPE (1,2-Dielaidoyl-sn-glycero-3-phosphoethanolamine) was purchased from NOF Co. Dim-23 was synthesized by VK Chemical Services (Rehovot, Israel). DSPE-PEG-mal, 1,2-distearoyl-sn-glycero-3-phosphoethanolamine-N-[maleimide(polyethylene glycol)-3400] (ammonium salt) was from Layson Bio Inc. Traut's reagents was obtained from Thermo Scientific. Mouse IgG and Rabbit anti-mouse IgG Fcy⁻ fragment specific were from Jackson Immuno Research Laboratories. Dil was purchased from Biotium Inc.

### In Vitro and In Vivo Stability Test of RBC/Lipid Anchor Conjugation

To study the stability of RBC/lipid anchor conjugation, balb/c female mouse blood was used for an in vitro test and balb/c female mice were used for an in vivo test. An example is listed as follows.

### Generation of Sulfhydryl Groups on IgG

Mouse IgG (1.02 mg) from was dissolved in 182 μl buffer (0.01 M sodium phosphate, 0.25 M NaCl, pH 7.6) at 5.6 mg/mL. A certain amount of Traut's Reagent solution (5 mg/mL in DPBS, 7.5 μl) and EDTA buffer (50 mM in DPBS, 19 μl) were added to the above IgG solution. The final concentration of EDTA in the mixture was 5 mM. The mixture was incubated for 1 h at room temperature (RT) on a shaker followed by filtering with a spin desalting column (Zeba, MWCO 7K, Thermo Scientific) based on the manufacturer's instructions to remove the unreacted Traut's reagent. The desalted solution was collected and ready for use. The sulfhydryl groups on the modified IgG were quantified using Ellman's Reagent following the manufacturer's protocol. Generally, the usage of 40-fold mole of Traut's reagent (equivalent to IgG) resulted in 1-2 sulfhydryl groups for each IgG.

### Coupling of IgG-SH and DSPE-PEG-mal

DSPE-PEG-mal (2 mM in DPBS, 6.8 μl) were added to the IgG-SH solution and incubated at RT on a shaker. After 1 hr, the sample solution was filtered using a centrifugal filter device (Microcon YM-50, 50K, Millipore Co.) at 14000 g for 15 min at 4° C. to remove the small molecules and suspended in 500 μl DPBS. The above step was repeated at least 3 times. Finally the purified sample was resuspended in 200 μl DPBS. The IgG concentration in the sample solution was evaluated by UV absorbance at 280 nm.

### Conjugation of Red Blood Cells (RBCs) and DSPE-PEG-IgG

The female balb/c mouse blood was used to prepare RBCs. Generally, 250 μl of whole blood was suspended in 1000 μl DPBS and spun at 1500 g for 30 sec. The washing steps were repeated 4 times. Finally, the RBCs were suspended in certain amount of DPBS at 4×10⁹/mL. An automated cell counter (Countess, Invitrogen) was used to measure the cell concentration. The conjugation of RBC/DSPE-PEG-IgG was prepared by mixing 385 μl RBCs suspensions, 1095 μl DPBS and 60 μl DSPE-PEG-IgG solution followed by incubating for 30 min at 37° C. The final IgG concentration was 0.2 mg/mL. The mixture was cooled for 5 min at RT, washed 3 times by DPBS (same as RBC preparation method) and resuspended in 1540 μl DPBS.

Alternatively, other buffers may be used for the preparation of the red blood cells. PIGCA may also be used, which is also available commercially. PIGCA can be prepared as 2 mM ATP, 3 mM GSH, 5 mM adenine, 100 mM sodium pyruvate, 100 mM Inosine, 100 mM NaH₂PO, 100 mM glucose, and 12% NaCl.

### Dil Labeling of RBC/DSPE-PEG-IgG Conjugation

The above RBC/DSPE-PEG-IgG conjugation was incubated with 7.7 μl Dil solution (1 mM in ethanol) for 1 hr at RT followed by washing with DPBS for 3 times. Finally the Dil-labeled RBC conjugation was resuspended in 150 μl DPBS.

### In Vitro Stability Test of RBC Conjugation

The in vitro stability was studied in 227 μl of whole balb/c female blood by adding 100 μl of the above RBC/DSPE-PEG-IgG conjugation. The mixture was incubated at 37° C. and the sampling was done at 5 min, 1 hr, 3 hr and 24 hr, respectively.

### Injection of RBC/DSPE-PEG-IgG/Dil Conjugation

The female balb/c mice were weighed (WMouse, g) and the whole blood of each mouse (VBlood) was calculated based on the following equation.

VBlood (mL)=WMouse (g)×0.1 (mL/g)

A certain amount of RBC conjugation was injected into mouse through the tail vein. The injection amount of RBC conjugation is 2% of total mouse body blood (the modified blood/whole body blood=2%). After injection, the sampling was done by taking around 30 μl of mouse blood at 5 min, 1 hr, 2 hr, 6 hr, 24 hr, 48 hr and 72 hr, respectively. The sample with Dil-only labeling (no lipid anchor) was used as a control.

### Characterization of RBC Conjugation by Microscopy and FACS

The blood samples (20 μl) taken from blood and mice were washed 3 times by DPBS and resuspended in 200 μl DPBS. Alexa Fluor 488 Goat anti-mouse IgG (2 mg/ml, 2 μl, Invitrogen) was added to label the lipid anchor by incubating at RT for 20 min on a shaker. After 3-times washing by DPBS, the labeled RBC conjugation was resuspended in 400 μl DPBS and visualized by microscopy (Nikon) using a glass slide. A flow cytometry (FACSCalibur, Becton Dickinson) was used to quantify the double-labeled RBC conjugation. The standard beads (LinearFlow™ Green Flow Cytometry Intensity Calibration Kit, Invitrogen) was used to calibrate the green fluorescent intensity and evaluate sample brightness.

### In Vitro Tumor Binding Study

To study the binding efficiency of RBC/lipid anchor with the target tumor cell, an in vitro cell culture system using human A549 was developed. A typical example is listed as followings.

### Generation of Sulfhydryl Groups on IgG Fcy⁻ Fragment

Rabbit anti-mouse IgG Fcy⁻ fragment (1.32 mg) was dissolved in 560 μl buffer (0.01 M sodium phosphate, 0.25 M NaCl, pH 7.6) at 2.4 mg/mL. A certain amount of Traut's Reagent solution (5 mg/mL in DPBS, 30 μl) and EDTA buffer (50 mM in DPBS, 65 μl) were added to the above IgG solution. The mixture was incubated for 1 h at room temperature (RT) on a shaker followed by filtering with a spin desalting column (Zeba, MWCO 7K, Thermo Scientific) following the manufacturer's instructions to remove the unreacted Traut's reagent. The desalted solution was collected and ready for use. The sulfhydryl groups on the modified IgG were quantified using Ellman's Reagent (Pierce) based on the manufacturer's protocol. Generally, the usage of 40-fold of Traut's reagent (molar equivalent to IgG) resulted in 1-2 sulfhydryl groups for each IgG.

### Coupling of Fcy⁻-SH and DSPE-PEG-mal

DSPE-PEG-mal (2 mM in DPBS, 27 μl) were added to the salted Fcy⁻-SH solution and incubated at RT on a shaker. After 1 hr, the sample solution was filtered using a centrifugal filter device (Microcon YM-50, 50K, Millipore Co.) at 14000 g for 15 min at 4° C. to remove the small molecules and suspended in 500 μl DPBS. The above step was repeated at least 3 times. Finally the purified sample was resuspended in 200 μl DPBS. The IgG fragment concentration in the sample solution was quantified by UV absorbance at 280 nm.

### Conjugation of Red Blood Cells (RBCs) and DSPE-PEG-Fcy⁻

The female balb/c mouse blood was used to prepare RBCs. Generally, 250 μl of whole blood was suspended in 1000 μl DPBS and spun at 1500 g for 30 sec. The washing steps were repeated 4 times. Finally, the RBCs were suspended in certain amount of DPBS at 4×10⁹/mL. An automated cell counter (Countess, Invitrogen) was used to measure the cell concentration. The conjugation of RBC/DSPE-PEG-Fcy⁻ was prepared by mixing 1000 μl RBCs suspensions, 2800 μl DPBS and the above DSPE-PEG-Fcy⁻ solution followed by incubating for 30 min at 37° C. The mixture was cooled for 5 min at RT, washed 3 times by DPBS (same as RBC preparation method) and resuspended in 4000 μl DPBS.

### Dil Labeling of RBC/DSPE-PEG-Fcy⁻ Conjugation

The above RBC/DSPE-PEG-Fcy⁻ conjugation was incubated with 20 μl Dil solution (1 mM in ethanol) for 1 hr at RT followed by washing with DPBS for 3 times. Finally the Dil-labeled RBC conjugation was resuspended in 4000 μl DPBS.

### Conjugation of A549 and Ep-CAM

A549 cell (2×10⁷/mL, 1000 μl) were incubated with 5 μl Ep-CAM (Alexa Fluor 488 anti-human CD326 Ep-CAM, Clone 9C4, Biolegend) for 1 hr at RT followed by washing 3 times and resuspending in 1000 μl DPBS.

Binding of RBC/DSPE-PEG-Fcy⁻ Conjugation with A549/Ep-CAM

The binding of RBC/A549 was performed by incubating 1000 μl DSPE-PEG-Fcy⁻ conjugation and 1000 μl A549/Ep-CAM conjugation for 2 hr at RT on a shaker. The samples were visualized by a fluorescent microscope.

### Example 2

### Antibody Constructs for Stable Incorporation into the Red Blood Cell Membrane

Various methods may be used to conjugate the ligand, such as an antibody, to the red blood cells. Examples include using lipid, lipopeptide, and transmembrane protein domain linkages. In certain embodiments, non-phospholipid lipids, which do not carry a phosphate charge, may be appropriate, as the non-phospholipid lipids may not change the overall charge of the membrane.

Lipid-antibody constructs are designed and synthesized to exhibit high incorporation efficiency into the red blood cell membrane without causing damage to the cells, while achieving stable association and long circulation life in the blood of the modified cells. Several methods for modification of the cell surface have been tested before, including direct conjugation of polyethylene glycol, immunoglobulins and enzymes [14, 15][16][17]. Some of these methods resulted in RBCs circulating as long as 55 days [15]. Phospholipid conjugates have been explored for incorporation of antibodies in the liposomal membrane [18]. Lipid conjugation is preferred over chemical conjugation to limit damage to the proteins, which severely limits circulation time of the cells in the body.

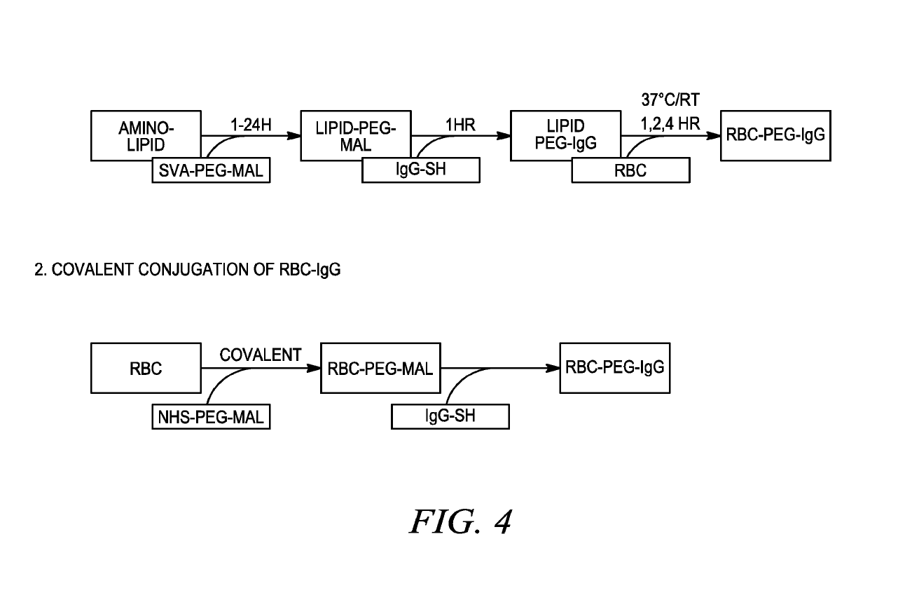

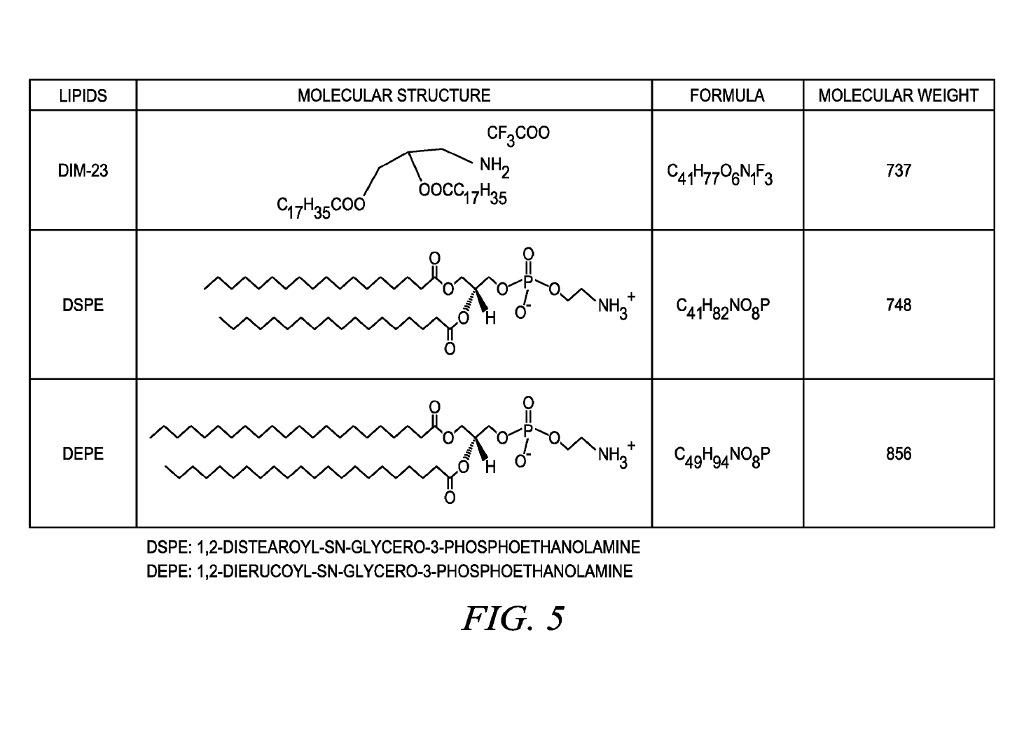

Lipid and lipopolymer chemistry was tested in order to achieve the most stable conjugation of the IgG to the cells. Whole IgG or shortened Fab portion may be used to avoid potential immune recognition of the RBC by body macrophages. The antibody was conjugated to lipid molecules using heterobifunctional PEG linkers. An example of a modification strategy is shown in FIG. 4. Various lipid-antibody constructs may be tested including phospholipids, single chained and multiple-chained lipids, for example having 2, 3, 4, 5, 6, 7, or 8 chains with different chain length and saturation, from C12 to C22, for example, having 12, 13, 14, 15, 16, 17, 18, 19, 20, 21, or 22 carbon, and different number of lipid molecules per antibody, such as, for example, 1, 2, 3, 4, 5, 6, 7, 8, 9, 10, 11, 12, 13, 14, 15, 16, 17, 18, 19, or 20 lipid molecules per antibody. In some examples, DSPE-antibody constructs (18-carbon lipid) are more highly retained within the RBC membrane than DPPE constructs (16-carbon lipid) over a 1 hour circulation period in a mouse model. Dextran DPPE constructs show, in certain examples, similar retention to that of DPPE without dextran backbone. Examples include, but are not limited to, those shown in FIG. 5: Dim-23, DSPE, and DEPE.

Different lengths of PEG linkers may be tested (Lysan Bio, Inc.). In addition, dextran lipopolymers [19] may be prepared. The immunoglobulins at various and IgG/dextran molar ratios are prepared. IgG conjugated on polymers may, for example, afford better association with the membrane. Lipopeptides, for example peptide GKGGKGGKGGKC, may be used. Lysines, for example, may be used for attaching lipids (single, double, or triple chain) and cysteine may, for example, be used for coupling the antibody. Peptide or polymer backbones may also be used for grafting the lipids.

For lipid-antibody (Ab) incorporation, different incubation conditions are tested, including incubation buffer, incubation time, temperature. The damage to the RBC is assessed. The efficiency of incorporation is determined by staining cells with fluorescently labeled anti-rabbit antibody and quantification of the cell fluorescence The absolute number of the antibody copies per cell is determined. The covalent chemical binding of IgG to the RBC membrane integral proteins may also be tested. Heterobifunctional polyethylene glycol is used to attach the thiolated antibody molecules to cell membrane. In addition, combination of lipid chemistry and activatable linker chemistry is explored to anchor first the Ab to the membrane and then “lock” it by covalent linkage.

The RBC incorporation efficiency may increase as a function of number and length of lipid chains. In certain examples, the number of conjugated IgG molecules is between 10⁴ and 10⁷ per cell. Cell shape and morphology should remain intact after incubation and washing steps. Intensive processing and modification of the RBCs may produce hemolysis or severe shape changes in the cells [14, 20]. Damage may result due to PEG interaction with cell membranes or detergent-like action of lipids. In that case, the labeling concentration is adjusted accordingly, including change of washing and conjugation buffer. Alternatively, if the labeling efficiency is low (which may be determined based on the levels of the staining with secondary antibody) the incubation conditions are adjusted. Further, the number of ligands, such as antibodies, per red blood cell may be adjusted to improve efficiency and activity.

Other methods for incorporating the ligand into the cell membrane include, for example, changing the lipid composition of the red blood cells by incubating the cells with phospholipid liposomes, and changing the lipid composition of the red blood cells by incubating the cells with methyl-beta-cyclodextrin, to remove cholesterol from the membrane, or cyclodextrin-cholesterol, for enriching the membrane with cholesterol.

Other methods may be used to link the ligand to the red blood cell, including covalent chemistry methods. These methods include, but are not limited to Azide-alkyne click chemistry, Azide-phosphine (Staudinger) chemistry, Heterobifunctional linker such as NHS-haloacetyl, NHS-maleimide, NHS-Pyridylthiol, Homobifunctional linker (amine to amine, thiol to thiol, carboxyl to carboxyl), and Photoreactive linker (e.g., NHS-diaziridine). In other examples, a combination of lipid anchor and photoreactive chemistry may be used, such as lipid anchor containing diaziridine. First, the lipid is anchored to the membrane, then UV is applied and the lipid is covalently attached to the membrane proteins.

Examples of PEG linkers include those from 2 ethylene oxide units to 200 units in length, such as for example, 1, 2, 3, 4, 5, 6, 7, 8, 9, 10, 20, 30, 40, 50, 60, 70, 80, 90, 100, 120, 140, 160, 180, or 200 units in length. Other linkers may be about the same length.

### Example 3

### Alpha v Beta 3 Integrin Cells

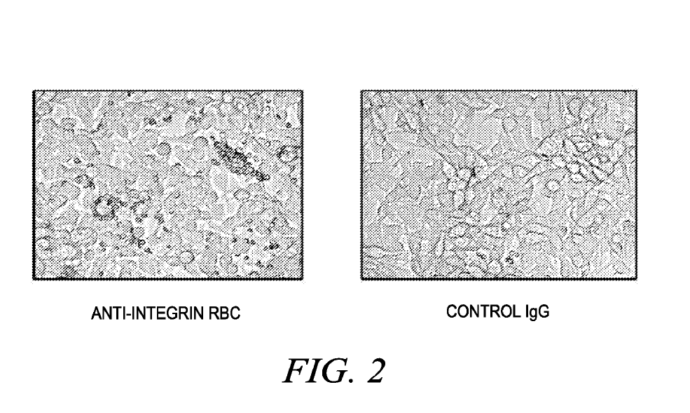

Anti-alpha v beta3 integrin was conjugated to PEG-DSPE (Avanti) using maleimide chemistry. The antibody molecules were conjugated on the RBC surface. The RBC showed strong binding to cancer cells in vitro. FIG. 2 depicts the binding of modified RBC to integrin expressing B16/F1 tumor cells grown in microscopy chambers. Black dots in the left image are RBCs that adhered to the cells.

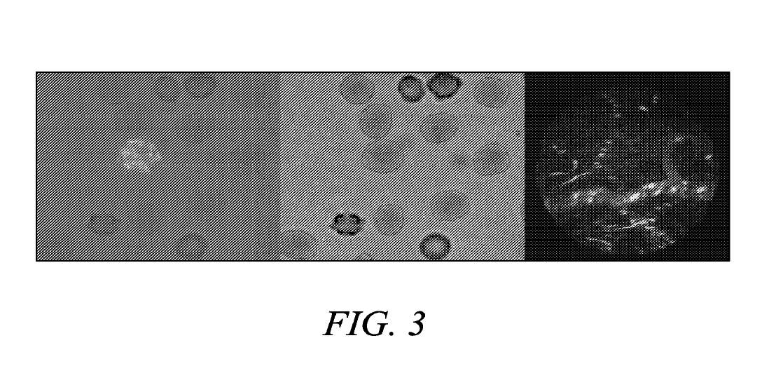

Next, the modified RBCs were injected into a Balb/C female mouse. Blood was sampled through the periorbital vein at 1 min, 24 h and 48 h post-injection. The cells were washed and stained with anti-rabbit IgG labeled with Alexa 488. According to FIG. 3, 48 hours post injection, almost 80% of the IgG conjugated RBCs were circulating, although their fluorescence intensity was somewhat decreased. The passage of FITC-labeled and Dil labeled cells was studied by intravital microscopy (FIG. 3) FIG. 3 depicts IgG coated RBC in mice. Left, fluorescence+transmitted light image; Center, transmitted light image. Right, real time microscopy showing FITC labeled and Dil labeled erythrocytes in the angiogenic vasculature (blue contrast due to Cy5 dextran).

### Example 4

### Testing and Optimizing the Efficiency of Incorporation In Vivo by Monitoring Circulation Time and Stability of the Conjugate

The circulation time and stability of the conjugated RBCs in vivo, and the conjugation protocol are optimized before administration to a patient. A longer circulation time would be advantageous because the cells will have a better chance of encountering the metastatic cells and interacting with the metastatic vasculature. The conjugation and manipulation of red blood cells can alter their circulation properties of the mice due to phagocytosis and liver/spleen extraction of damaged erythrocytes. Thus, damaged and oxidized cells are recognized by scavenger receptors in the liver [21] while fragile RBCs become trapped and destroyed in the spleen [22]. The stability of the association of the lipid conjugate with the cell membrane is also of importance. While most of the lipids are sufficiently stable in the membrane, the rate of exchange of the lipid is dictated by length and lipid solubility of the lipid chain and the polar part. With the large protein, the rate of exchange could be increased because of high critical micelle concentration.

Different antibody constructs prepared in Example 1 are incorporated into RBCs, which are intravenously injected into BALB/c mice. In addition, the cells are labeled with Dil in order to independently monitor the RBC clearance. Blood samples are collected from periorbital vein at different time points and the blood cells are washed and stained with the secondary Ab against the conjugated IgG. The level of fluorescence per cell before and after injection is quantified. Two parameters are determined: half-life of cells in the circulation and half-life of the IgG on the cell surface. These factors may only partially related to each other. The data points are plotted against time and the half-lives may be calculated using Prism software.

A blood half-life between, for example, 3 and 21 days may be found using the conjugation methods. The stability of the IgG on the membrane is expected to be in the similar range. Coating with immunoglobulins can theoretically enhance macrophage recognition through Fc-gamma receptor and complement receptors [23]. In the case of short circulation time and fast exchange rate of the lipid-Ab conjugate are observed, the lipid formulation or/and the conjugation protocol are adjusted. Conjugates with Fab part of the antibody and shorter peptides (single-chain fragment) instead of full-length IgG are tested to circumvent this issue.

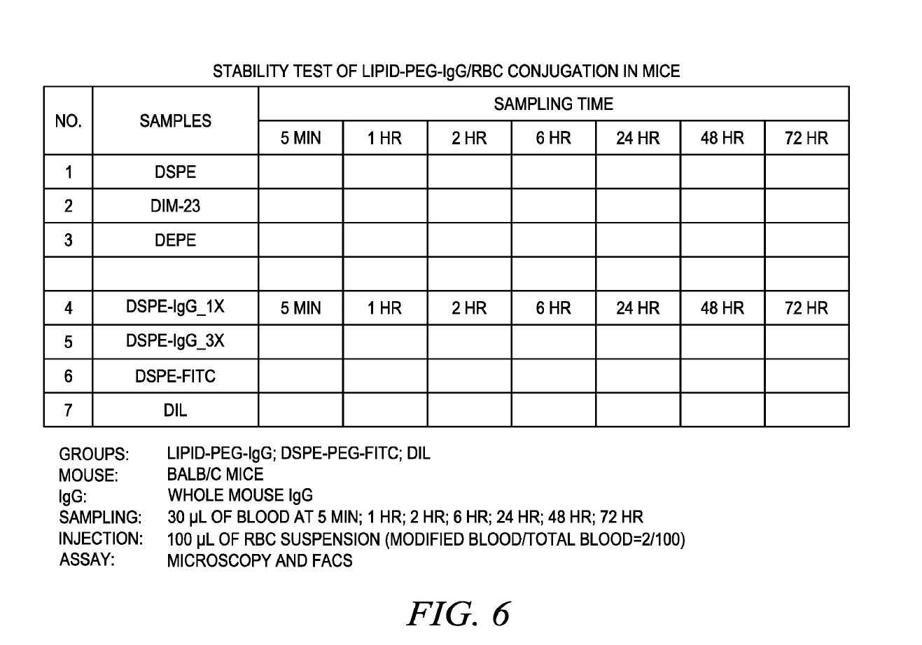

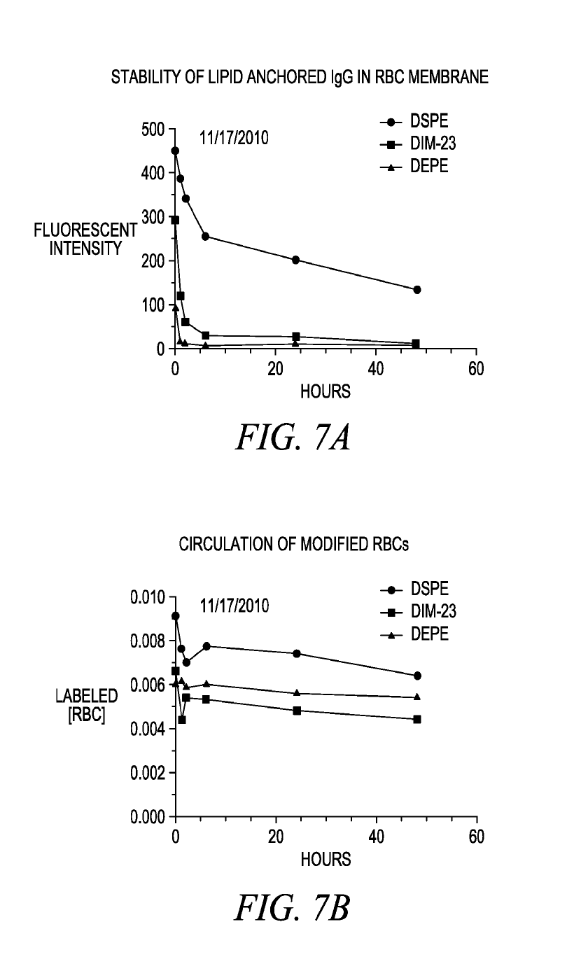

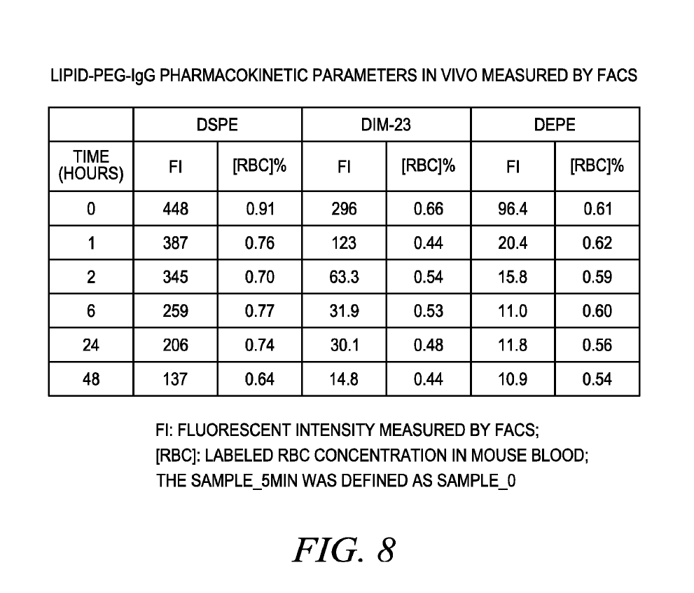

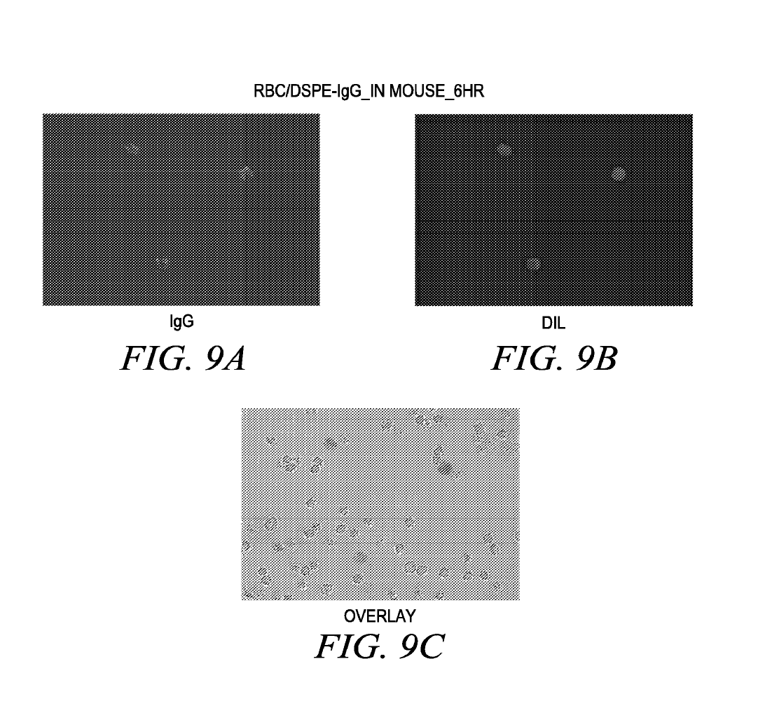

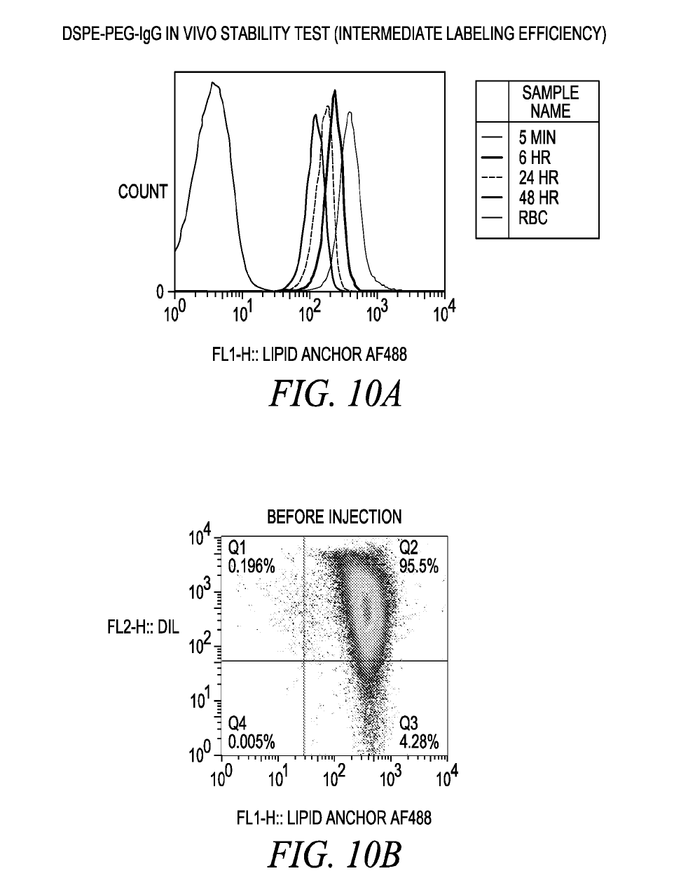

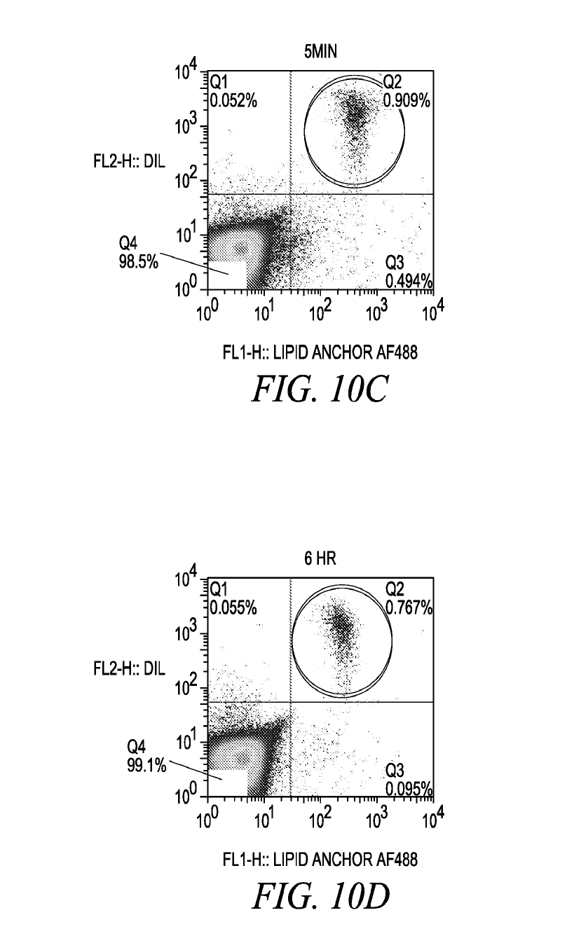

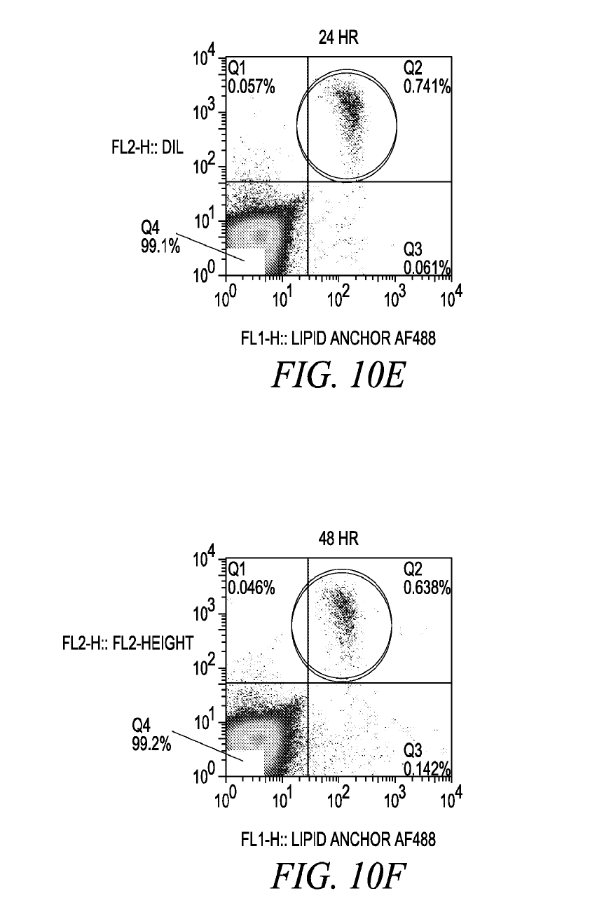

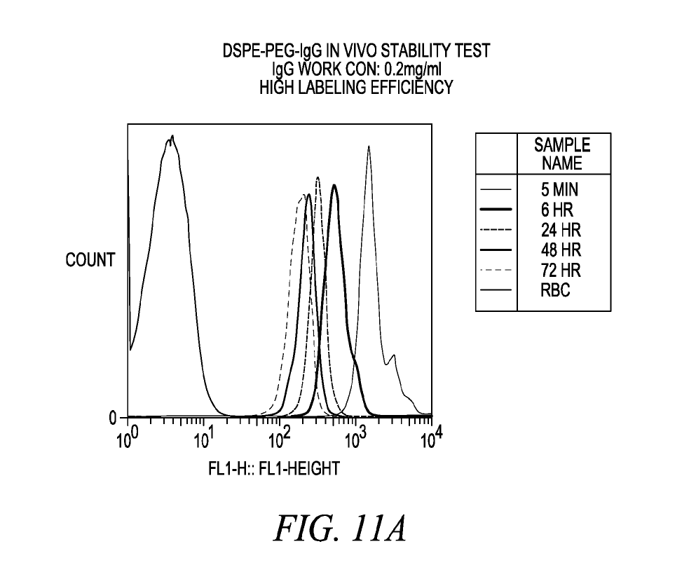

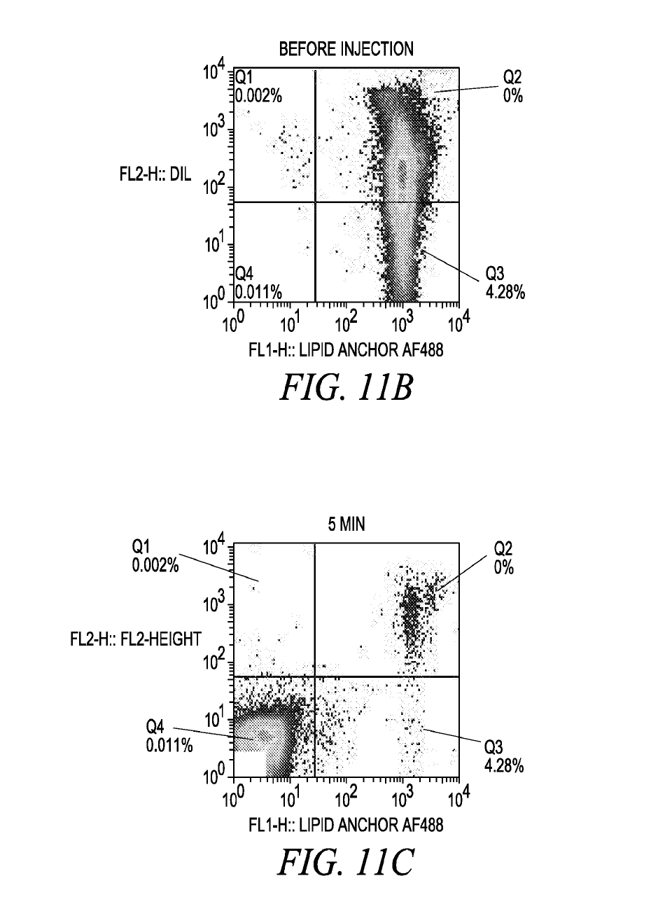

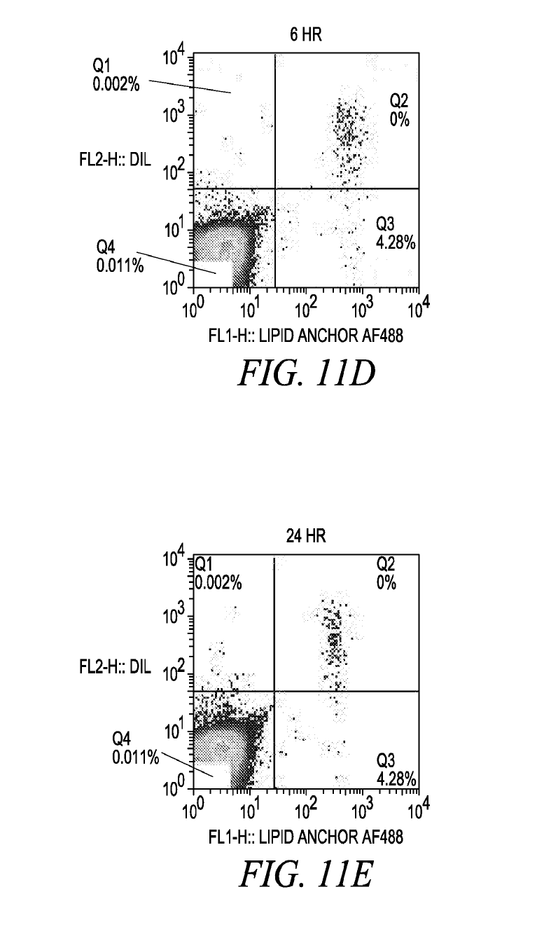

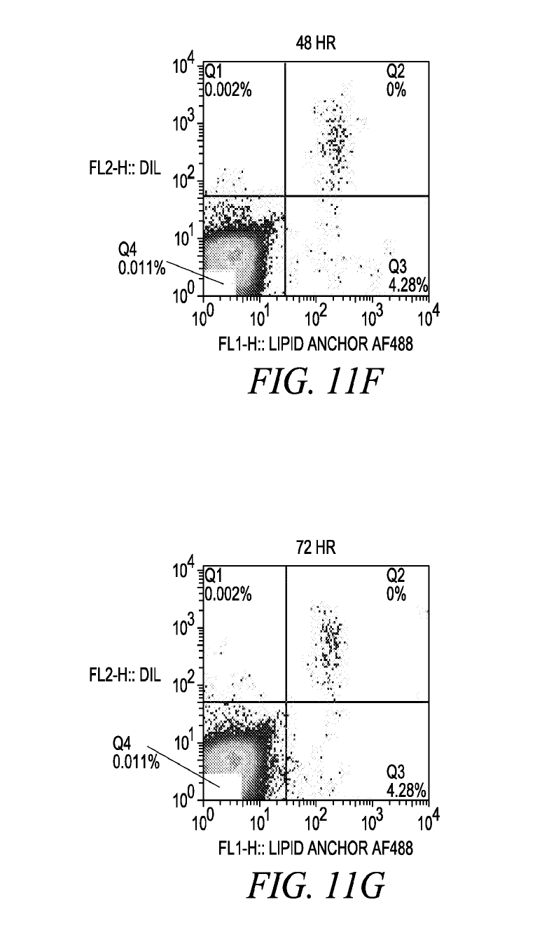

A sample stability test of various antibody-lipid conjugated red blood cells, in mice, is presented in FIG. 6. 100 microliters of an antibody-lipid conjugated red blood cell suspension (modified blood/total blood=2/100) was injected into BalbC mice. 30 microliter blood samples were collected at 5 minutes, 1 hour, 2 hours, 6 hours, 24 hours, 48 hours, and 72 hours. Detection was performed using microscopy and FACS. Results of the stability assay using lipid-IgG and Dil are shown in FIG. 7. FIG. 8 presents the pharmacokinetic parameters of the in vivo test, as measured by FACS. FIG. 9 presents photos of a blood smear obtained 6 hours after red blood cells modified with IgG and Dil were injected into BalbC mice. The photos show that IgG is retained in the red blood cell membrane. FIGS. 10 and 11 show the results using DSPE-PEG-IgG in mice. FIG. 10 shows longevity and stability of IgG lipid construct that was inserted at intermediate concentration, while FIG. 11 shows the RBCs that were labeled with high IgG lipid concentration. Over labeling the cells (FIG. 11) causes shortening of RBC survival and negatively affects the retention of the lipid in the membrane. Without limiting the scope of the technology, this could be due to the excessive membrane modification, or aggregation and clumping of individual lipid molecules on the membrane. The solution could be in using different lipid or the way they are attached to the Ab. The DSPE-PEG linker was found to have a half-life over 72 hours in mice.

The longevity of the red blood cells and the lipid conjugate may depend on the concentration of the ligand in the membrane, its chemical properties, and the protocol used to conjugate the lipids to the red blood cells. Red blood cells that have a more concentrated lipid conjugate in the membrane, and red blood cells where the lipid conjugate is more hydrophobic, are less stable. These conditions may be modified and tested using methods in the art.

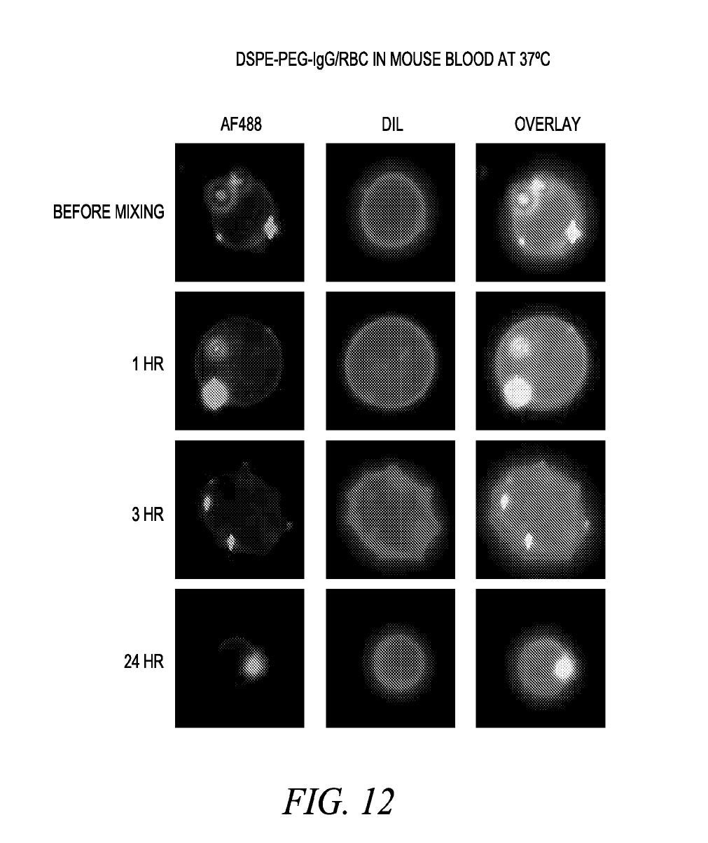

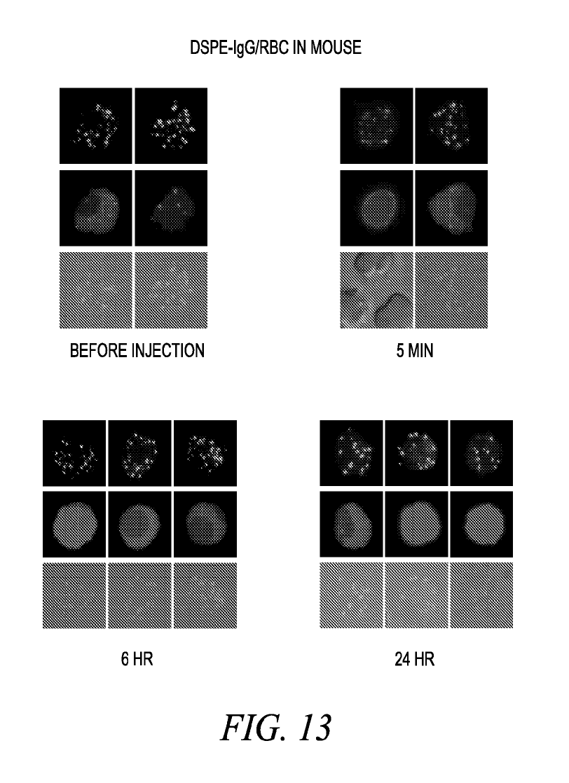

Red blood cells labeled with the marker Dil and IgG, were incubated in whole mouse blood for up to 24 hours. This in vitro test, shown in FIG. 12, correlates with the in vivo test in that the lipid anchored IgG is relatively stable. FIG. 13 shows the results of an in vivo stability test in which the coated red blood cells DSPE-IgG/RBC) were injected in mice and samples were taken at various time points, as indicated, and the fluorescence of the sample was observed.

### Example 5

### Test the Binding of Modified Erythrocytes to Cells in Culture

The modification of RBCs that result in high affinity binding of to target cells, such as metastatic tumor cells and angiogenic endothelium cells is confirmed by methods such as those presented in this example. During attachment to the cells in vivo, the cells will experience shear force [24, 25]. The stability of the antibody-lipid construct in the membrane of the RBCs should be high enough to withstand shear stress in the bloodstream. Usually, the attachment of cells under shear stress is studied using parallel flow chamber or controlled shear flow microfluidic system [7]. These systems together with plain mixing are used to assess the stability of attachment between RBC and tumor cells.

The ability to bind different types of the cells is assessed. The adherent 4T1 and MDA-MB-231 breast carcinoma cells that express epithelial cell adhesion molecule EpCAM [26] are grown on tissue plate and may be detached using a scraper before the experiment. RBCs are modified with the anti EpCAM antibody. The binding is determined by incubation in Thermomixer™ (Eppendorf) at 37° C. for 30 min and 60 min and counting the percentage of tumor cells that are associated with the RBCs. In a similar fashion, endothelial HUVEC cells are used to test binding of RBCs to alpha v beta 3 integrin. In another set of experiments a microfluidic device developed at UCSD may be used to test the strength of adhesion of the RBCs to cells [7].

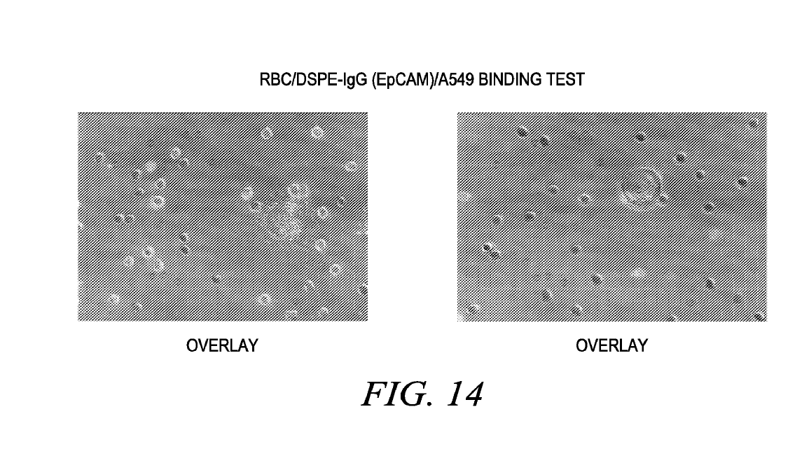

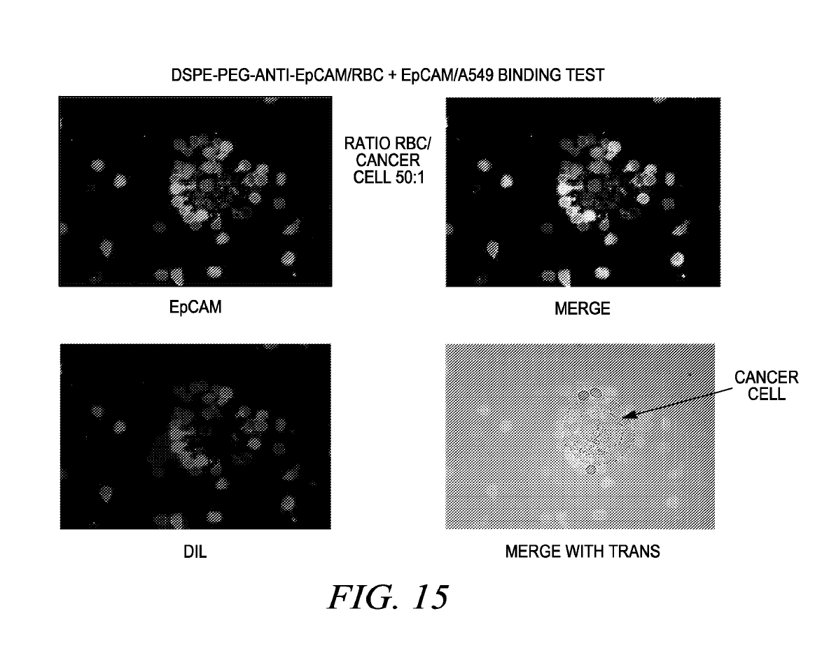

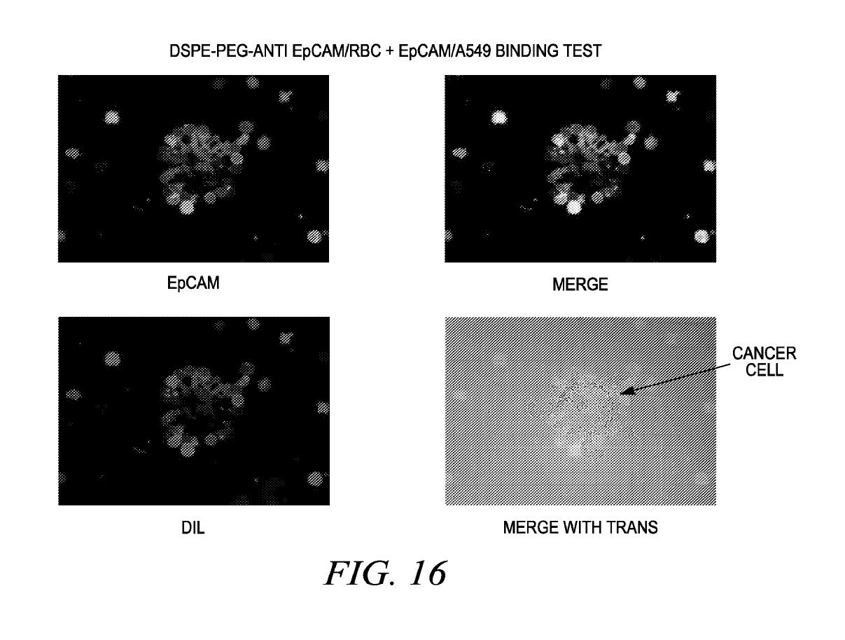

FIG. 14 presents the results of a binding study in which red blood cells were modified with anti-EpCAM antibody and incubated with A549 lung carcinoma cells. The binding of the red blood cells to the carcinoma cells was studied by microscopy. FIG. 15 shows the results of a binding assay in which DSPE-PEG-anti-EpCAM/red blood cells were incubated in vitro for 30 minutes with EpCAM/A549 cells. The cancer cells are not labeled in the photos. In FIG. 15 D, an A549 cell is almost completely coated with the labeled red blood cells.

The binding of RBC is expected to be strong enough so that only small percentage of RBCs will dissociate from the target cells. Should the stability of the binding be lower than 80% after 1 h incubation (vortexing), the stability of binding is adjusted through the number of antibody molecules per RBC.

### Example 6

### Test the Binding of the Modified RBC to Tumor Cells and Angiogenic Vasculature in Metastatic Breast Cancer Models

The biological fate of modified RBCs after injection into circulation and the biological fate of the primary cancer or metastatic tumor cells when they become associated with RBCs in the bloodstream is tested. Numerous tools for the study of growth and invasion of human and mouse metastases have been developed using fluorescent whole body imaging and intravital video microscopy [6,7]. These tools may be used to assess the aspects of the modified RBC action.

Intravital fluorescent microscopy and whole body fluorescent imaging are used in order to monitor distribution of the metastatic cells and RBC in the tissues. Tumor cells with GFP label are injected via intravenous or intraportal routes. For the study of metastatic cell delivery and arrest in liver vasculature, the cells are injected via portal vein and observed at different times using live imaging microscopy as described [6,7]. The red blood cells modified with anti-EpCAM, anti-αᵥβ3 integrin or control antibody are either preinjected prior to the tumor cells or mixed with the cells and injected together. The colocalization between the tumor cell and the RBC may suggest their association in the blood stream.

Blood samples are taken at various time points and the association between the cells is monitored. In parallel, different organs such as liver, spleen, kidney and bone marrow are imaged to observe the pattern of metastatic cell distribution following attachment to RBCs. The difference in the distribution may be quantified by counting the events. In addition, the association between the tumor cells and RBC is studied. Liver, spleen and kidney and lung, are removed and disintegrated, and GFP tumor cells are counted to monitor their biodistribution and degree of colocalization as the result of RBCs.

For entrapment of the RBC in the neovasculature and the subsequent blockade, red fluorescent protein expressing metastatic tumors may be implanted under the skin of GFP-positive mice and the Cy5 labeled modified RBC may be injected intravenously. Intravital microscopy may be used to study and quantify the binding of RBCs to angiogenic vasculature and changes in blood flow.

The following scenarios are possible: (a) formation of tumor cell-RBC complex that could be also associated with platelets and leukocytes; (b) Entrapment of the complexes in the microvasculature of different organs; (c) Prevention of extravasation of the tumor cells bound to the RBCs (d) entrapment of the tumor cell-RBC complex by the spleen and liver macrophages with subsequent destruction; (e) attachment of the cells to the angiogenic capillaries and staunching the blood flow there.

Potential negative outcomes are possible, such as detachment of the tumor cells from RBCs and then extravasation in target organ, or extravasation of the whole RBC-tumor cells complex. It is unlikely that attached erythrocytes would contribute to the arrest of the cancer cells in the capillaries because of the flexibility and much smaller size of the RBCs. However, it is not known how these events will affect the development of metastasis, therefore controlled treatment study is warranted.

### Example 7

### Test the Effect of Modified RBCs on Metastatic Colonization and Growth Using Mouse Models

This pilot study helps to determine if there is any therapeutic benefit from the use of the modified RBC in prevention of colonization and growth of metastases in mouse models. It is not clear to what extent the binding of erythrocytes to tumor vasculature and metastatic cells will prevent extravasation and how much the decreased extravasation will affect tumor growth. Similarly, the contribution of the macrophages in the decrease of the metastatic growth is not clear.

4T1 cells, which originally derived from a spontaneous mouse mammary tumor of a BALB/C mouse, grow rapidly when injected into the fat pad of a syngeneic animal and metastasize to lungs, liver, bone, and brain. This model in part resembles the multiple stages involved in malignant breast cancer development in patients. MDA-MB-231 cells, an estrogen-independent breast cancer cell line derived from the pleural effusion of a cancer patient, is able to colonize bone, liver, lung, adrenal glands, ovary, and brain after intravenous injection. The direct introduction of cancer cells into the blood circulation is considered an assay of organ colonization and not a true metastatic process.

GFP-expressing or luciferase-expressing 4T1 tumors are grown in the mammary fat pad. Modified or control RBC or PBS are injected at time intervals after the main tumor grows beyond 1 cm. Alternatively, cells are injected together with RBCs into the mammary fat pad, to test if the RBCs prevent formation of the main tumor. After one-two weeks the mice are studied for metastatic growth using, for example, whole body luciferase imaging (Xenogen) or organ fluorescent imaging. The incidence of the metastases in organs may be quantified by image intensity or by counting metastatic foci. The depletion of cells from bone marrow is studied. A control group may include mice injected with plain non-conjugated antibodies

In a different study, 4T1 or MDA MB-231 cells expressing GFP or luciferase is injected intravenously. With this route of administration, mostly lung metastases may develop. The mice are preinjected with modified or control RBC or PBS and additional boluses are injected throughout the study. In addition, RBC and tumor cells are mixed together and injected intravenously. The incidence of metastases is studied as described for the orthotopic model. Long-circulating RBCs are expected to decrease the metastatic process by, for example, at least 50% with the colonization model and at least 25% in the spontaneous growth model. In the worst case scenario, number of metastases will not be reduced or that organ distribution of metastases will change due to the entrapment and arrest of RBC-tumor cell complexes in highly vascularized organs. This latter scenario is unlikely as the location of metastases is determined mostly by the permissive tissue microenvironment and only to minor extent by physical entrapment in the vasculature.

### REFERENCES

Citations referred to in the present application, and providing further technical support.
1. Parker B, Sukumar S. Distant metastasis in breast cancer: molecular mechanisms and therapeutic targets. Cancer Biol Ther 2003; 2(1):14-21.
2. Punglia R S, Morrow M, Winer E P, Harris J R. Local therapy and survival in breast cancer. N Engl J Med 2007; 356(23):2399-405.
3. Cameron D. Lapatinib plus capecitabine in patients with HER2-positive advanced breast cancer. Clin Adv Hematol Oncol 2007; 5(6):456-8.
4. Lin N U, Dieras V, Paul D, Lossignol D, Christodoulou C, Stemmler H J, et al. Multicenter phase II study of lapatinib in patients with brain metastases from HER2-positive breast cancer. Clin Cancer Res 2009; 15(4):1452-9.
5. Miller K, Wang M, Gralow J, Dickler M, Cobleigh M, Perez E A, et al. Paclitaxel plus bevacizumab versus paclitaxel alone for metastatic breast cancer. N Engl J Med 2007; 357(26):2666-76.
6. Yamauchi K, Yang M, Jiang P, Xu M, Yamamoto N, Tsuchiya H, et al. Development of real-time subcellular dynamic multicolor imaging of cancer-cell trafficking in live mice with a variable-magnification whole-mouse imaging system. Cancer Res 2006; 66(8):4208-14.
7. Gutierrez E, Groisman A. Quantitative measurements of the strength of adhesion of human neutrophils to a substratum in a microfluidic device. Anal Chem 2007; 79(6):2249-58.
8. Wiedswang G, Borgen E, Schirmer C, Karesen R, Kvalheim G, Nesland J M, et al. Comparison of the clinical significance of occult tumor cells in blood and bone marrow in breast cancer. Int J Cancer 2006; 118(8):2013-9.
9. Cristofanilli M, Budd G T, Ellis M J, Stopeck A, Matera J, Miller M C, et al. Circulating tumor cells, disease progression, and survival in metastatic breast cancer. N Engl J Med 2004; 351(8):781-91.
10. Baker M K, Mikhitarian K, Osta W, Callahan K, Hoda R, Brescia F, et al. Molecular detection of breast cancer cells in the peripheral blood of advanced-stage breast cancer patients using multimarker real-time reverse transcription-polymerase chain reaction and a novel porous barrier density gradient centrifugation technology. Clin Cancer Res 2003; 9(13):4865-71.
11. Weidner N. Tumoural vascularity as a prognostic factor in cancer patients: the evidence continues to grow. J Pathol 1998; 184(2):119-22.
12. Osta W A, Chen Y, Mikhitarian K, Mitas M, Salem M, Hannun Y A, et al. EpCAM is overexpressed in breast cancer and is a potential target for breast cancer gene therapy. Cancer Res 2004; 64(16):5818-24.
13. Gasparini G, Brooks P C, Biganzoli E, Vermeulen P B, Bonoldi E, Dirix L Y, et al. Vascular integrin alpha(v) beta3: a new prognostic indicator in breast cancer. Clin Cancer Res 1998; 4(11):2625-34.
14. Hashemi-Najafabadi S, Vasheghani-Farahani E, Shojaosadati S A, Rasaee M J, Armstrong J K, Moin M, et al. A method to optimize PEG-coating of red blood cells. Bioconjug Chem 2006; 17(5):1288-93.
15. Scott M D, Murad K L, Koumpouras F, Talbot M, Eaton J W. Chemical camouflage of antigenic determinants: stealth erythrocytes. Proc Natl Acad Sci USA 1997; 94(14):7566-71.
16. Taylor R P, Sutherland W M, Reist C J, Webb D J, Wright E L, Labuguen R H. Use of heteropolymeric monoclonal antibodies to attach antigens to the C3b receptor of human erythrocytes: a potential therapeutic treatment. Proc Natl Acad Sci USA 1991; 88(8):3305-9.
17. Ganguly K, Krasik T, Medinilla S, Bdeir K, Cines D B, Muzykantov V R, et al. Blood clearance and activity of erythrocyte-coupled fibrinolytics. J Pharmacol Exp Ther 2005; 312(3):1106-13.
18. Moghimi M, Moghimi S M. Lymphatic targeting of immuno-PEG-liposomes: evaluation of antibody-coupling procedures on lymph node macrophage uptake. J Drug Target 2008; 16(7):586-90.
19. Azzam T, Eliyahu H, Shapira L, Linial M, Barenholz Y, Domb A J. Polysaccharide-oligoamine based conjugates for gene delivery. J Med Chem 2002; 45(9):1817-24.
20. Tragner D, Csordas A. Biphasic interaction of Triton detergents with the erythrocyte membrane. Biochem J 1987; 244(3):605-9.
21. Willekens F L, Werre J M, Kruijt J K, Roerdinkholder-Stoelwinder B, Groenen-Dopp Y A, van den Bos A G, et al. Liver Kupffer cells rapidly remove red blood cell-derived vesicles from the circulation by scavenger receptors. Blood 2005; 105(5):2141-5.
22. Valk P E, Guille J. Measurement of splenic function with heat-damaged RBCs: effect of heating conditions: concise communication. J Nucl Med 1984; 25(9):965-8.
23. Walport M J, Peters A M, Elkon K B, Pusey C D, Lavender J P, Hughes G R. The splenic extraction ratio of antibody-coated erythrocytes and its response to plasma exchange and pulse methylprednisolone. Clin Exp Immunol 1985; 60(3):465-73.
24. Simon S I, Goldsmith H L. Leukocyte adhesion dynamics in shear flow. Ann Biomed Eng 2002; 30(3):315-32.
25. Bell G I. Models for the specific adhesion of cells to cells. Science 1978; 200(4342):618-
26. Amann M, Brischwein K, Lutterbuese P, Parr L, Petersen L, Lorenczewski G, et al. Therapeutic window of MuS110, a single-chain antibody construct bispecific for murine EpCAM and murine CD3. Cancer Res 2008; 68(1):143-51.
27. Chambers A F, Groom A C, MacDonald I C. Dissemination and growth of cancer cells in metastatic sites. Nat Rev Cancer 2002; 2(8):563-72.
28. Hamidi, M., Zarrin, a., Foroozesh, M., Mohammadi-Samini S. Applications of carrier erythrocytes in delivery of biopharmaceuticals. J. Control Release 2007; 118:145-60.
29. Magnani, M., Rossi, L., Fraternale, A., Bianchi, M., Antonelli, M., Crinelli, R., and Chiarantini, L. Erythrocyte-mediated delivery of drugs, peptides and modified oligonucleotides. Gene Therapy 2002; 9:749-51.
30. Patel, P D, Dand, N., Hirlekar, R S, and Kadam, V. J. Drug-loaded erythrocytes: As novel drug delivery system. Current Pharmaceutical Design 2008; 14:63-70.
31. Jain, S., and Jain, N K. Engineered nanoerythrocytes as a novel drug delivery system. Erythrocyte Engineering for Drug Delivery and Targeting, Ed. Mauro Magnani. Springer, 2003. 77-92.
32. McNeel, D G, Eickhoff, J., Lee, Ft. et al., Phase I trial of a monoclonal antibody specific for alphavbeta3 integrin (MEDI-522) in patients with advanced malignancies, including an assessment of effect on tumor perfusion. Clin. Cancer. Res. 2005; 11:7851-60.
33. Urbich, C., and Dimmeler, S., Endothelial progenitor cells: Characterization and role in vascular biology. Circulation Research 2004; 95:343-53.
34. Yin, A., et al. AC133, a novel marker for human hematopoietic stem and progenitor cells. Blood 1997; 90:5002-5012.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

1-60. (canceled)

61. A method for inhibiting the dissemination of cancer cells in a patient, comprising contacting the cells with a red blood cell linked to a cancer cell-specific ligand.

62. The method of claim 61, wherein the cancer cells are primary cancer cells or metastatic cancer cells.

63. The method of claim 61, wherein the cell-specific ligand is an antibody, a peptide, or a small molecule.

64. The method of claim 61, wherein the ligand is conjugated to a lipid, a lipopeptide, or a transmembrane protein domain, and the conjugated ligand is incorporated into the cell membrane of the red blood cell.

65. The method of claim 61, wherein the ligand is an antibody that binds to an antigen selected from the group consisting of prostate specific membrane antigen, carcinoembryonic antigen, integrin alpha v beta 3, integrin alpha v beta 5, EpCAM, CD133, nucleolin, VEGF receptor 1 and VEGF receptor 2.

66. The method of claim 61, wherein the ligand is covalently linked to a molecule on the cell membrane of the red blood cell.

67. The method of claim 61, wherein the ligand is conjugated to a lipid.

68. The method of claim 61, wherein the cancer cells and the red blood cells linked to ligands form cell complexes.

69. A method for inhibiting the growth of neovasculature in a patient comprising administering to the patient a red blood cell linked to an angiogenic cell targeting ligand or an endothelial progenitor cell targeting ligand.

70. The method of claim 69, wherein the ligand is an antibody, a peptide, or a small molecule.

71. The method of claim 69, wherein the ligand adheres to early angiogenic capillaries.

72. The method of claim 69, wherein the neovasculature is associated with a tumor.

73. The method of claim 72 wherein the growth of the tumor is inhibited after administering the red blood cell to the patient.

74. A composition comprising a red blood cell linked to a cancer cell-specific ligand.

75. The composition of claim 74, wherein the ligand is an antibody, a peptide, or a small molecule.

76. The composition of claim 74, wherein the ligand is an antibody.

77. The composition of claim 76, wherein the antibody is an anti-angiogenic cell antibody.

78. The composition of claim 76, wherein the antibody adheres to early angiogenic capillaries.

79. The composition of claim 76, wherein the antibody is conjugated to a lipid, a lipopeptide, or a transmembrane protein domain, and the conjugated antibody is incorporated into the cell membrane of the red blood cell.

80. The composition of claim 76, wherein the antibody binds to an antigen selected from the group consisting of prostate specific membrane antigen, carcinoembryonic antigen, integrin alpha v beta 3, integrin alpha v beta 5, EpCAM, CD133, nucleolin, VEGF receptor 1 and VEGF receptor 2.
